(19) United States
(12) Patent Application Publication
(10) Pub. No.: US 2018/0214129 A1
(43) Pub. Date: Aug. 2, 2018

(54) MEDICAL IMAGING APPARATUS

(71) Applicant: KONINKLIJKE PHILIPS N.V., EINDHOVEN (NL)

(72) Inventors: CECILE DUFOUR, EINDHOVEN (NL); BENOIT JEAN-DOMINIQUE BERTRAND MAURICE MORY, Eindhoven (NL); GARY CHENG-HOW NG, EINDHOVEN (NL)

(21) Appl. No.: 15/505,626

(22) PCT Filed: Sep. 7, 2015
(86) PCT No.: PCT/EP2015/070362

§ 371 Date: Feb. 22, 2017

(30) Foreign Application Priority Data
Sep. 8, 2014 (EP) 14306376.6

(51) Int. Cl. A61B 8/00; G06T 7/11; A61B 8/08

(52) U.S. Cl. CPC A61B 8/466; A61B 8/4245; G06T 7/11; A61B 8/5261; A61B 8/4483; A61B 8/4416; G06T 2207/10136; G06T 2207/10088; A61B 8/469; A61B 8/4444; G06T 2207/10081

(57) ABSTRACT

A medical imaging apparatus (10) for evaluating medical image data is disclosed. The medical imaging apparatus comprises an ultrasound acquisition unit including an ultrasound probe (14) for acquiring ultrasound image data of a patient (12) and an ultrasound segmentation unit (24) for segmenting anatomical features of the patient in the ultrasound image data and for providing ultrasound segmentation data (46). The apparatus comprises an image data interface (18) for receiving 3D medical image data of the patient and a medical image segmentation unit (26) for segmenting the 3D medical image data and for providing medical image segmentation data (48). A user input interface (38) is provided for identifying a position (44) by the user in the 3D medical image data and/or in the ultrasound image data in order to initiate the segmentation of anatomical features by the medical image segmentation unit and/or the ultrasound segmentation unit on the basis of the position identified by the user, wherein a registration unit (32) correlates the ultrasound segmentation data and the medical image segmentation data.

### FIELD OF THE INVENTION

The present invention relates to a medical imaging apparatus for evaluating medical image data. The present invention further relates to a medical image evaluation method for evaluating medical image data and to a computer program comprising program code means for causing a computer to carry out the steps of the method for evaluating medical image data.

### BACKGROUND OF THE INVENTION

In the field of medical imaging systems, it is generally known to combine different images of a patient acquired by different medical analysis systems in order to improve the diagnostic possibilities. In particular, ultrasound systems are known which combine ultrasound images and preoperative image data of a patient derived from a different analytic system like MRT or CT. To enable the fusion of live ultrasound images of a patient with the preoperative volume data of the same patient, a position tracking system is usually utilized to spatially align the different image data.

The position tracking systems rely on a calibration e.g. based on artificial markers which can be identified in the preoperative and the ultrasound data and which can be correlated to each other so that the alignment of the data can be determined.

Further, the alignment of the different image data can be based on automatic registration of anatomical features like vessels identified in the different image data, however, the automatic registration is complex, involves large technical effort and is not reliable for any case. A corresponding system for automatically correlating images from different imaging systems is e.g. known from US 2013/0053679 A1.

The position tracking system may also be calibrated on the basis of a user input, wherein a plurality of corresponding positions are identified by the operator in both image data to be aligned. However, this method needs an expert as an operator to calibrate the position tracking system, so that this system is cumbersome.

Physics in Medicine and Biology, vol. 57, no 1, 29 Nov. 2011, pages 81-91, discloses an automatic registration between 3D intra-operative ultrasound and pre-operative CT images of the liver.

### SUMMARY OF THE INVENTION

It is therefore an object of the invention to provide an improved medical imaging apparatus and a corresponding improved medical imaging evaluation method for evaluating medical image data, which is more reliable and less complicated for the user.

According to one aspect of the present invention, a medical imaging apparatus is provided for evaluating medical image data, comprising:
an ultrasound acquisition unit including an ultrasound probe for acquiring ultrasound image data of a patient,
an ultrasound segmentation unit for segmenting anatomical features of the patient in the ultrasound image data and for providing ultrasound segmentation data,
an image data interface for receiving 3D medical image data of the patient, a medical image segmentation unit for segmenting the 3D medical image data and for providing medical image segmentation data,
a user input interface for identifying a position by the user in the 3D medical image data and/or in the ultrasound image data in order to initiate the segmentation of anatomical features by the medical image segmentation unit and/or the ultrasound segmentation unit on the basis of the position identified by the user,
a registration unit for correlating the ultrasound segmentation data and the medical image segmentation data.

According to another aspect of the present invention, a medical image evaluation method is provided for evaluating medical image data, comprising the steps of:
acquiring ultrasound data of a patient by means of an ultrasound probe,
receiving 3D medical image data of the patient,
identifying a position in the 3D medical image data and/or in the ultrasound image data by a user via a user input interface,
segmenting anatomical features of the patient in the ultrasound data and providing ultrasound segmentation data of the anatomical features,
segmenting anatomical features the 3D medical image data and providing medical image segmentation data,

wherein the segmentation of the anatomical features in the ultrasound data and/or the 3D medical image data is initiated on the basis of the position identified by the user, and
correlating the ultrasound segmentation data and the medical image segmentation data.

According to still another aspect of the present invention, a computer program is provided comprising program code means for causing a computer to carry out the steps of the medical image evaluation method according to the present invention, when said computer program is carried out on a computer.

Preferred embodiments of the invention are defined in the dependent claims. It shall be understood that the claimed method has similar and/or identical preferred embodiments as the claimed device and as defined in the dependent claims.

The present invention is based on the idea that a position in the 3D medical image data and/or the ultrasound image data is identified by the user via a user input interface in order to provide the information to the system which anatomical features are considered to be advantageous for a segmentation and for a registration of the different image data. The segmentation of the anatomical features performed by the medical image segmentation unit and/or the ultrasound segmentation unit are initiated on the basis of the identified position so that the segmentation unit does not need to perform a segmentation of the whole image data and the technical effort and the calculation time is significantly reduced. On the basis of the segmentation data which is calculated on the basis of the identified position, the registration unit can register the ultrasound image data and the 3D medical image data so that a tracking of the ultrasound probe and/or or a fusion of the different image data can be performed with high accuracy. Since the position identified by the user initiates the position of the segmentation, the effort for segmenting the ultrasound image data and/or the 3D medical image data can be significantly reduced and the reliability of the registration can be improved since the most significant anatomical features of the image data can be easily identified by the user input and the influence of artifacts can be reduced.

Consequently, the present invention achieves an evaluation of medical image data with improved reliability and which is comfortable for the user.

In a preferred embodiment, the position identified by the user is a point in the 3D medical image data and/or the ultrasound image data. In a further preferred embodiment, the position identified by the user corresponds to a voxel of the 3D medical image data and/or a voxel or a pixel of the ultrasound image data.

In a preferred embodiment, the medical imaging apparatus further comprises a position determining unit attached to the ultrasound probe for determining the position of the ultrasound probe, wherein the position determining unit includes a calibration unit for calibrating the position of the ultrasound probe on the basis of the correlation of the segmentation data received from the registration unit. This is a possibility to reduce the evaluation effort during a surgery, since the position determining unit can further improve the registration if it is calibrated on the basis of the correlation of the segmentation data.

In a further preferred embodiment, the medical imaging apparatus further comprises a fusion unit for fusion of the ultrasound image data and the 3D medical image data on the basis of the position of the ultrasound probe determined by the position determining unit. This is a possibility to provide a continuously fused medical image on the basis of the ultrasound image data and the 3D medical image data.

The fusion unit may also alternatively or additionally to the fusion of the ultrasound image data and the 3D medical image data on the basis of the position of the ultrasound probe adapted to fuse the ultrasound image data and the 3D medical image data on the basis of the correlation of the ultrasound segmentation data and the medical image segmentation data provided by the a registration unit. This is a possibility to improve the fusion of the ultrasound image data and the 3D medical image data.

The fusion of the ultrasound image data and the 3D medical image data is performed by the fusion unit continuously during the acquisition of the ultrasound image data so that a fused image based on the combination of the ultrasound image data and the 3D medical image data can be provided in real time.

In a preferred embodiment, the 3D medical image segmentation unit is adapted to segment anatomical features in the 3D medical image data adjacent to or surrounding the position defined in the 3D medical image data. This is a possibility to utilize segmentation data of certain anatomical features which can be easily identified so that the reliability of the registration can be improved.

In a preferred embodiment, the ultrasound segmentation unit is adapted to segment anatomical features in the ultrasound image data adjacent to or surrounding the position identified in the ultrasound image data. This is a possibility to initiate the segmentation of certain anatomical features which can be easily identified in the ultrasound image data so that the reliability of the registration can be improved.

In a preferred embodiment, the anatomical features are surfaces in the vicinity of the identified position. This is a possibility to improve the accuracy of the registration, since surfaces in the image data can be easily identified by means of the segmentation unit.

In a preferred embodiment, the anatomical features are vessels of the patient. This is a possibility to further improve the registration, since the shape of the vessels can be easily identified by the segmentation unit and the unique shape of the vessels can be easily registered so that the reliability of the correlation of the different image data can be improved.

In a preferred embodiment, the ultrasound segmentation unit and a medical image segmentation unit are adapted to determine centre lines and/or bifurcations of the vessels and wherein the registration unit is adapted to register the ultrasound image data and the 3D medical image data on the basis of the determined centre lines and/or bifurcations of the vessels. This is a possibility to reduce the technical effort for the registration of the image data and to improve the accuracy of the registration, since the centre lines and the bifurcations can be easily derived from the segmentation data and the so derived data can be registered with high accuracy and low technical effort.

In a preferred embodiment, the user input interface comprises a display unit for displaying the 3D medical image data and/or the ultrasound image data and wherein the user interface comprises an input device for identifying the position of the 3D medical image data and/or the ultrasound image data at the display unit. This is a possibility to easily identify the position in the image data so that the user input is more comfortable.

In a preferred embodiment, the input device is adapted to control a position of an indicator displayed at the display unit within the displayed image data and to identify the position in the displayed image on the basis of the position of the indicator and a user input. This is a further possibility to identify the position with high precision and low effort for the user, since the indicator is displayed at the display unit within the displayed image data. In a further preferred embodiment, the indicator is a mouse pointer or the like and the input device comprises an input unit like a mouse or the like, wherein the position can be identified by a single mouse click within the displayed image data. This is a possibility to further improve the comfort of the user input and to reduce the effort for the user.

In a further preferred embodiment, the display unit comprises a contact sensitive surface for identifying the position in the displayed image by a user input. In other words, the display unit is formed as a touchscreen, wherein the position in the image data is identified by a single touch at the corresponding position displayed at the display unit. This is a possibility to further improve the accuracy of the identification of the position and to reduce the effort for the user.

In a preferred embodiment, the 3D medical image data is previously acquired and the image data is stored in a memory device. This is a possibility to combine medical image data of different analysis methods which can be captured of the patient prior to the ultrasound analysis so that the examination time can be reduced.

In a preferred embodiment, the 3D medical image data is MR image data, CT image data, cone-beam CT image data or ultrasound image data. These are possibilities to improve the diagnostic possibilities, since the different analysis methods have different contrasts and different identification techniques and the amount of information of the anatomical features can be improved.

As mentioned above, the present invention can improve the reliability of the registration, since the segmentation is based on the position identified by the user or initiated on the basis of the identified position and the technical effort, and in particular the calculation effort can be reduced, since the system does not need to provide segmentation data of the whole image data since the region of interest is identified by the user input. Further, since the operator merely needs to identify one position in the image data and does not need to identify corresponding positions in the different image data, no expert knowledge is necessary and the handling is more comfortable for the user.

### BRIEF DESCRIPTION OF THE DRAWINGS

These and other aspects of the invention will be apparent from and elucidated with reference to the embodiment(s) described hereinafter. In the following drawings

FIG. 1 shows a schematic representation of a medical imaging apparatus in use to scan a volume of a patient's body;

FIG. 2a, b show an ultrasound image and a CT image of a certain site of the patient's body to be correlated;

FIG. 3a, b show the images of FIG. 2a, b partially segmented to register the image data;

FIG. 4a, b show segmentation data of the vessels of the image data shown in FIG. 3a, b;

FIG. 5a, b show centre lines and bifurcations derived from the segmentation data shown in FIG. 4a, b;

FIG. 6 shows a correlation of the centre lines and the bifurcations identified in the segmentation data;

FIG. 7a, b show the initial ultrasound image shown in FIG. 2a and the fused ultrasound and CT image fused on the basis of the segmentation and registration procedure; and

FIG. 8 shows a flow diagram of a method for evaluating medical image data.

### DETAILED DESCRIPTION OF THE INVENTION

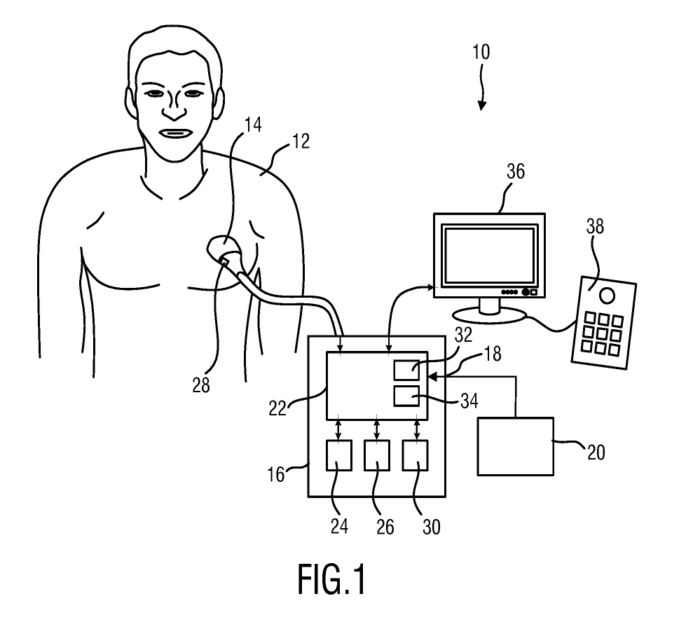

FIG. 1 shows a schematic illustration of a medical imaging apparatus generally denoted by 10. The medical imaging apparatus 10 is applied to inspect a volume of an anatomical site, in particular an anatomical site of a patient 12. The medical imaging apparatus 10 comprises an ultrasound probe 14 having at least one transducer array including a multitude of transducer elements for transmitting and receiving ultrasound waves. The transducer elements are preferably arranged in a 2D array, in particular for providing multi-dimensional image data.

A medical imaging apparatus 10 comprises in general an image processing apparatus 16 connected to the ultrasound probe 14 for evaluating the ultrasound data received from the ultrasound probe 14 and for combining or correlating the ultrasound images with preoperative images of the patient 12. The imaging processing apparatus 16 comprises an image interface 18 for receiving the preoperative 3D medical image data from a database 20 or an external analysis and imaging apparatus 20. The preoperative image data is preferably computer tomography image data (CT), magnetic resonance tomography image data (MRT), cone-beam CT image data or preoperative 3D ultrasound image data. The image processing apparatus 16 comprises an image processing unit 22 connected to the ultrasound probe 14 and to the image interface 18 for evaluating the ultrasound data and for providing ultrasound image data from the volume or object of the patient 12 which is analyzed by the ultrasound probe 14 and for evaluating the preoperative 3D medical image data received from the image interface 18.

The image processing apparatus 16 further comprises an ultrasound segmentation unit 24 for segmenting anatomical features of the patient in the ultrasound image data and for providing a corresponding ultrasound segmentation data to the image processing unit 22. The image processing apparatus 16 further comprises a medical image segmentation unit 26 for segmenting the 3D medical image data received from the database 20 via the interface 18 and for providing medical image segmentation data to the image processing unit 22.

The medical imaging apparatus 10 further comprises a position determining unit 28 attached to the ultrasound probe 14 for determining a position of the ultrasound probe 14. The position determining unit 28 determines the relative position of the ultrasound probe, e.g. by means of electromagnetic tracking in order to determine a movement of the ultrasound probe 14 with respect to an initial or a calibrated position. The initial position is calibrated by means of a calibration unit 30. The calibration unit 30 is connected to the image processing unit 22 in order to correlate the ultrasound data captured by the ultrasound probe 14, the position of the ultrasound probe 14 received from the position determining unit 28 and the 3D medical image data on the basis of the ultrasound segmentation data and medical image segmentation data received from the ultrasound segmentation unit 24 and the medical image segmentation unit 26 as described in the following. The so determined position of the ultrasound probe 14 with respect to the 3D medical image data is used as a reference position or used as calibrated position of the ultrasound probe 14. If the ultrasound probe 14 is moved with respect to the calibrated position, the position determining unit 28 detects the distance and the direction with respect to the calibrated position and provides a so determined current position of the ultrasound probe 14.

The image processing unit 22 further comprises a registration unit 32 for correlating the ultrasound segmentation data and the medical image segmentation data. The calibration unit 30 calibrates the position of the ultrasound probe 14 with the respective ultrasound data and the 3D medical image data of the patient 12 on the basis of the correlation of the ultrasound segmentation data and the medical image segmentation data received from the registration unit 32.

The image processing unit 22 further comprises a fusion unit 34 for fusion of the ultrasound image data and the 3D medical image data on the basis of the position of the ultrasound probe 14 determined by the position determining unit 28.

The fusion unit 34 may also utilize the correlation of the ultrasound segmentation data and the medical image segmentation data received from the registration unit 32 in order to fuse the ultrasound image data and the 3D medical image data. This is a possibility to further improve the fusion of the different data.

The medical imaging apparatus 10 further comprises a display unit 36 for displaying image data received from the image processing apparatus 16. The display unit 36 receives the image data in general from the image processing unit 22 and is adapted to display the ultrasound image data and the 3D medical image data and also the respective segmentation data. The medical imaging apparatus 10 further comprises an input device 38 which may be connected to the display unit 36 or to the image processing apparatus 16 in order to control the image acquisition and to identify a position in the 3D medical image data and/or in the ultrasound image data displayed on the display unit 36. The input device 38 may comprise a keyboard or a mouse or the like or may be formed as a touchscreen of the display unit 36 to identify or indicate a certain anatomical feature or a position within the displayed ultrasound image data and/or the 3D medical image data.

The image processing unit 22 is adapted to receive the position identified in the image data by means of the input device 38 by the user. The image processing unit 22 initiates on the basis of the position identified in image data the ultrasound segmentation unit 24 or the medical image segmentation unit 26 to perform a segmentation of the respective image data at the identified position and in the vicinity of the identified position and/or surrounding the identified position.

The imaging processing unit 22 and in particular the registration unit 32 comprised in the image processing unit 22 correlates the ultrasound segmentation data and the 3D medical image segmentation data and the fusion unit 34 combines the respective ultrasound image data and the 3D medical image data to provide a composed medical image and provides the composed medical image to the display unit 36.

Since the segmentation of an anatomical feature of the patient 12 is initiated by the position determined by the user within the displayed ultrasound image data and/or the 3D medical image data, the respective segmentation unit 24, 26 performs the segmentation at a certain anatomical feature which can be easily identified by the segmentation unit so that the technical effort and the calculation time for the segmentation is reduced and the anatomical features for the correlation can be identified faster and with an improved reliability.

The spatial alignment of the ultrasound image data and the 3D medical image data is performed by the fusion unit 34 on the basis of the correlation received from the registration unit 32 .

The fusion of the ultrasound image data and the 3D medical image data is performed by the fusion unit 34 continuously during the ultrasound scan. The fused image based on the combination of the ultrasound image data and the 3D medical image data can therefore be provided in real time during the ultrasound scan.

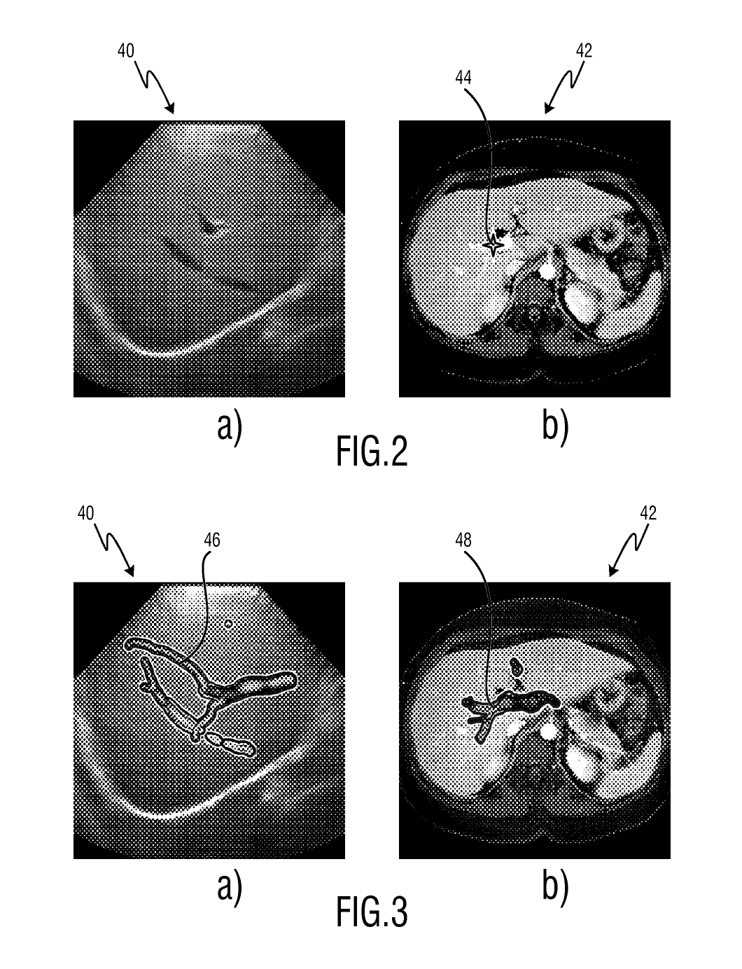

FIG. 2a shows an ultrasound image 40 on the basis of the ultrasound image data received from the ultrasound probe and captured from the patient 12. FIG. 2b shows a sectional medical image 42 on the basis of the 3D medical image data of the patient 12. In this particular case, FIG. 2a and FIG. 2b show the liver of the patient 12, wherein the ultrasound image 40 and the sectional medical image 42 are not yet spatially aligned or correlated to each other.

To initiate the segmentation in the sectional medical image 42, the user identifies a position in the sectional medical image 42 by means of the input device 38 as a user input interface. In FIG. 2b, the position is identified by an indicator 44 movable within the sectional medical image 42. The indicator 44 shows the position identified by the user, which is in this particular case the portal vein of the liver which is also visible in the field of view of the ultrasound image 40.

On the basis of the identified position, the segmentation of the 3D medical image data is initiated as shown in FIG. 3b and also the segmentation of the vessels is performed in the ultrasound image data as shown in FIG. 3a. Since the portal vein is identified by the user input and the indicator 44, the segmentation of this anatomical feature can be performed faster and with a higher reliability so that the overall reliability of the registration and correlation of the respective image data is improved. It shall be understood that the position of a certain anatomical feature can be identified within the sectional medical image 42 and/or in the ultrasound image 40 so that the segmentation in general can be performed faster and with a higher reliability.

The anatomical features surrounding the identified position are segmented, wherein the anatomical features may be surrounding surfaces like the vessels or other anatomical surfaces within the patient's body. The ultrasound segmentation data is in FIG. 3a denoted by 46 and the medical image segmentation data is in FIG. 3b denoted by 48.

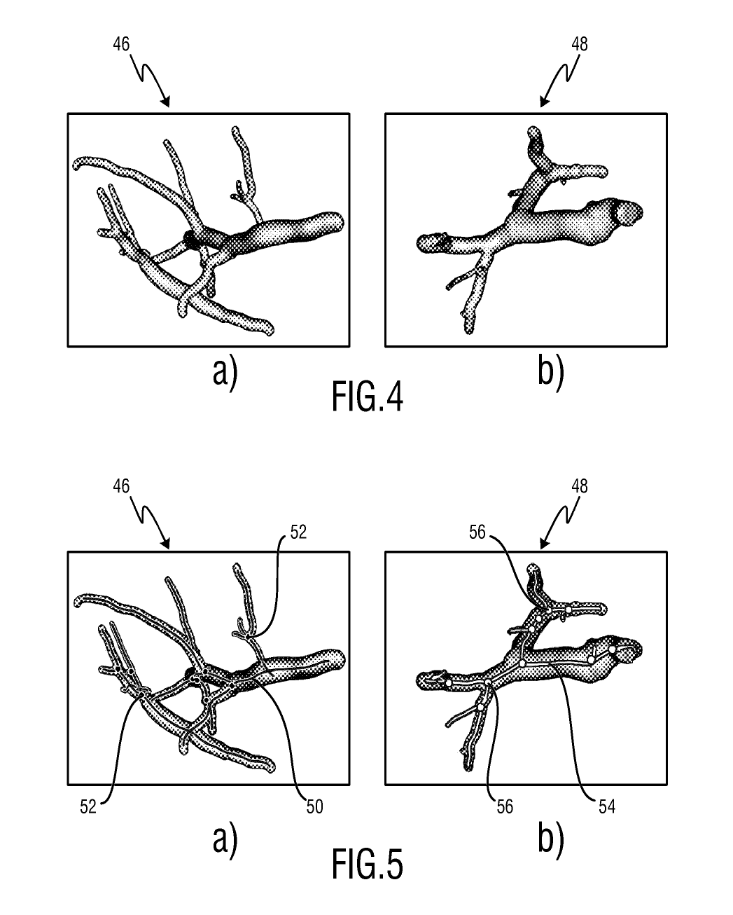

FIG. 4a shows the ultrasound segmentation data 46 of the vessels derived from the ultrasound image data by means of the ultrasound segmentation unit 24. FIG. 4b shows the medical image segmentation data derived from the 3D medical image data by means of the medical image segmentation unit 26.

The ultrasound segmentation unit 24 determines centre line 50 and bifurcations 52 from the ultrasound segmentation data 46 and the medical image segmentation unit 26 determines from the medical image segmentation data 48 centre lines 54 and bifurcations 56 as shown in FIG. 5b.

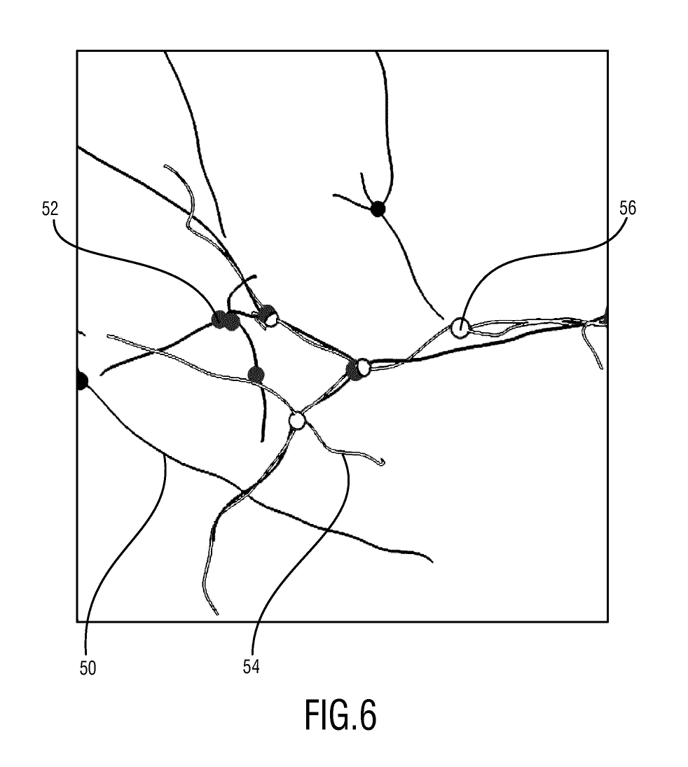

The registration unit 32 correlates the centre lines 50, 54 and the bifurcations 52, 56 of the segmentation data 46, 48 as shown in FIG. 6 and the fusion unit 34 combines the ultrasound image data and the 3D medical image data on the basis of the correlation received from the registration unit 32 and/or the position of the ultrasound probe 14 determined by the position determining unit 28.

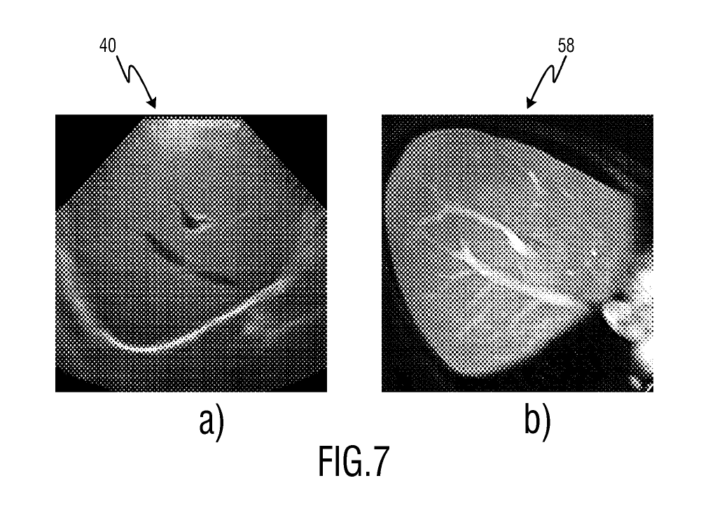

FIG. 7a shows the ultrasound image 40 shown in FIG. 2a and FIG. 7b shows an image spatially aligned by the fusion unit 34 of the image processing unit 22 on the basis of the correlation received from the registration unit 32 . The correlation can be easily performed by the user input, since the segmentation effort is reduced and the reliability of the identification of significant anatomical features within the image data can be improved.

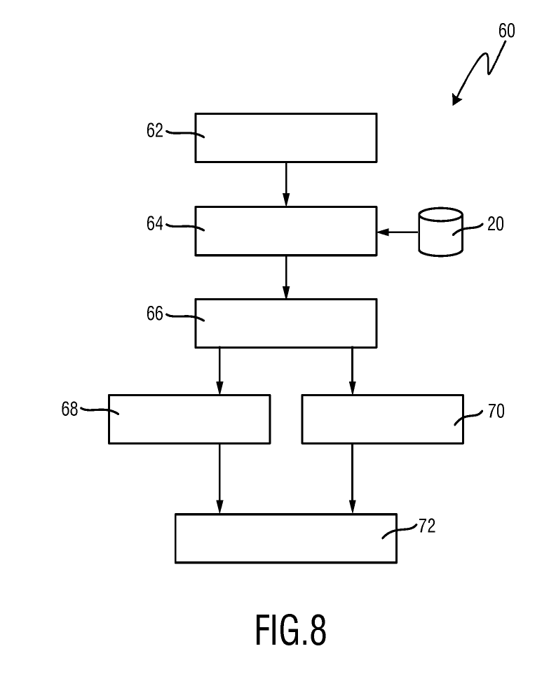

In FIG. 8 a flow diagram of a method for evaluating medical image data is shown and generally denoted by 60.

The method 60 starts with acquiring ultrasound data of the patient 12 by means of the ultrasound probe 14 as shown at step 62 and with receiving 3D medical image data of the patient 12 from the external database 20, which is usually MRT or CT data previously acquired from the patient 12 as shown at step 64. At step 66 a position is identified in the 3D medical image data and/or the ultrasound image data by the user via the input device 38 as shown at step 66.

The anatomical features of the patient 12 are segmented in the ultrasound data and corresponding segmentation data of the anatomical features are provided as shown at step 68. Further, anatomical features in the 3D medical image data are segmented and the medical image segmentation data 48 is provided as shown at the step 70. The segmentation of the anatomical feature in the ultrasound data and/or in the 3D medical image data are based on the identified position, wherein the respective segmentation in the ultrasound data and/or the medical image data are initiated on the basis of the identified position.

Preferably, the anatomical features surrounding the identified position are segmented in order to segment only those anatomical features which are relevant and which are identified by the user. If the position is identified in the 3D medical image data, the segmentation of the ultrasound data may be performed in general or if the position is identified in the ultrasound image data, the segmentation of the medical image data may be performed in general. In a certain embodiment, the position to be segmented is identified in the 3D medical image data as well as the ultrasound image data.

The ultrasound segmentation data and the medical image segmentation data are provided to the registration unit 32, wherein the ultrasound segmentation data 46 and the medical image segmentation data 48 are correlated at step 72.

On the basis of the so correlated segmentation data, the calibration of the position determining unit 28 can be performed and the fusion of the ultrasound image data and the 3D medical image data can be performed by the fusion unit 34.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. A medical imaging apparatus for evaluating medical image data, comprising:
an ultrasound acquisition unit including an ultrasound probe for acquiring ultrasound image data of a patient,
an ultrasound segmentation unit for segmenting anatomical features of the patient in the ultrasound image data and for providing ultrasound segmentation data,
an image data interface for receiving 3D medical image data of the patient,
a medical image segmentation unit for segmenting the 3D medical image data and for providing medical image segmentation data,
a user input interface arranged to identify a position input by the user in the 3D medical image data and/or in the ultrasound image data in order to initiate the segmentation of anatomical features by the medical image segmentation unit and/or the ultrasound segmentation unit on the basis of the position identified by the user, wherein said segmentation of anatomical features is performed in the data adjacent to or surrounding the identified position in the respective image data,
a registration unit for correlating the ultrasound segmentation data and the medical image segmentation data.

2. The medical imaging apparatus as claimed in claim 1, further comprising a position determining unit attached to the ultrasound probe for determining the position of the ultrasound probe, and a calibration unit for calibrating the position of the ultrasound probe on the basis of the correlation of the segmentation data received from the registration unit.

3. The medical imaging apparatus as claimed in claim 2, further comprising a fusion unit for fusion of the ultrasound image data and the 3D medical image data on the basis of the position of the ultrasound probe determined by the position determining unit.

4. (canceled)

5. (canceled)

6. The medical imaging apparatus as claimed in claim 1, wherein the anatomical features are surfaces in the vicinity of the identified position.

7. The medical imaging apparatus as claimed in claim 6, wherein the anatomical features are vessels of the patient.

8. The medical imaging apparatus as claimed in claim 7, wherein the ultrasound segmentation unit and the medical image segmentation unit are adapted to determine centre lines and/or bifurcations of the vessels and wherein the registration unit is adapted to register the ultrasound image data and the 3D medical image data on the basis of the determined centre lines and/or bifurcations of the vessels.

9. The medical imaging apparatus as claimed in claim 1, wherein the user input interface comprises a display unit for displaying the 3D medical image data and/or the ultrasound image data and wherein the user interface comprises an input device for identifying the position in the 3D medical image data and/or the ultrasound image data at the display unit.

10. The medical imaging apparatus as claimed in claim 9, wherein the input device is adapted to control a position of an indicator displayed at the display unit within the displayed image data and to identify the position in the displayed image data on the basis of the position of the indicator and a user input.

11. The medical imaging apparatus as claimed in claim 9, wherein the display unit comprises a contact sensitive surface for identifying the position the displayed image data by a user input.

12. The medical imaging apparatus as claimed in claim 1, wherein the 3D medical image data is previously acquired image data stored in a memory device.

13. The medical imaging apparatus as claimed in claim 1, wherein the 3D medical image data is MR image data, CT image data, cone-beam CT image data or ultrasound image data.

14. A medical image evaluation method for evaluating medical image data, comprising the steps of:
acquiring ultrasound data of a patient,
receiving 3D medical image data of the patient,
identifying a position in the 3D medical image data and/or in the ultrasound image data by a user via a user input interface,
segmenting anatomical features of the patient in the ultrasound data and providing ultrasound segmentation data of the anatomical features,
segmenting anatomical features the 3D medical image data and providing medical image segmentation data,
wherein the segmentation of the anatomical features in the ultrasound data and/or the 3D medical image data is initiated on the basis of the position identified by the user and performed in the data adjacent to or surrounding the identified position in the respective image data, and
correlating the ultrasound segmentation data and the medical image segmentation data.

15. A computer program comprising program code means for causing a computer to carry out the steps of the method as claimed in claim 14, when said computer program is carried out on a computer.
